SEQUENCE LISTING (10 sequences)

SEQ ID NO: 1 - DNA, 348 bp, Homo sapiens
gtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggtgacctccaagtgtggctcattaggcaacatccatcataaaccag

SEQ ID NO: 2 - DNA, 441 bp, Homo sapiens
gtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccag

SEQ ID NO: 3 - DNA, 19 bp, Artificial
  Forward primer for amplifying human tau, htauR1_F.
gaacctgaagcaccagccg

SEQ ID NO: 4 - DNA, 18 bp, Artificial
  Reverse primer for amplifying human tau, htauR3_R.
tcaggtcaactggtttgt

SEQ ID NO: 5 - DNA, 23 bp, Artificial
  Intercept primer for amplifying human 4Rtau, htauR2_R.
gacgtgtttgatattatcctttg

SEQ ID NO: 6 - DNA, 15 bp, Artificial
  Human 3Rtau recognition probe, 3RVIC.
aggtgcaaatagtct

SEQ ID NO: 7 - DNA, 17 bp, Artificial
  Human 4Rtau recognition probe, 4RFAM.
tagcaacgtccagtcca

SEQ ID NO: 8 - DNA, 19 bp, Artificial
  Forward primer for amplifying mouse tau, MMtauR1_F.
gaacctgaagcaccagcca

SEQ ID NO: 9 - DNA, 18 bp, Artificial
  Reverse primer for amplifying mouse tau, MMtauR3_R.
tcaggtccaccggcttgt

SEQ ID NO: 10 - DNA, 23 bp, Artificial
  Intercept primer for amplifying mouse 4Rtau, MMtauR2_R
gacgtgtttgatattatccttcg

(19) United States
(12) Patent Application Publication
(10) Pub. No.: US 2010/0261166 A1
(43) Pub. Date: Oct. 14, 2010

(54) METHOD FOR SPECIFICALLY AND SENSITIVELY AMPLIFYING A TARGET SEQUENCE

(76) Inventors: Ohoshi Murayama, Saitama (JP); Hitomi Ueno, Tokyo (JP)

Correspondence Address: FOLEY AND LARDNER LLP;SUITE 500, 3000 K STREET NW, WASHINGTON, DC, 20007, US

(21) Appl. No.: 12/159,414

(22) PCT Filed: Dec. 28, 2006
(86) PCT No.: PCT/JP2006/326200

§ 371 Date: Jun. 27, 2008

(30) Foreign Application Priority Data
Dec. 28, 2005 (JP) 377704/2005

(51) Int. Cl. C12Q 1/68; C12P 19/34

(52) U.S. Cl. 435/6; 435/91.2

(57) ABSTRACT

The present invention aims to develop a method for amplifying only a target gene while reducing the noise from a non-target gene having a similar sequence, as well as to develop a new method of quantitation based on acquisition of the signal from the amplified sequence with high precision (in high sensitivity and specificity).

As a result of their intensive studies, the present inventors revealed that the stated object could be attained by incorporating a third primer in the PCR reaction mixture and this has led to the accomplishment of the present invention.

### TECHNICAL FIELD

The present invention relates to a method for specifically and sensitively amplifying a target sequence.

### BACKGROUND ART

With the recent remarkable advances in the research technology in the field of biochemistry, it has become possible to detect details of a phenomenon that is only slightly taking place within a sample. For example, the use of a fluorescent dye has made it possible to review details of the ionic distribution within cells or assay the association of one protein with another. Included among those technical advances in the field of biochemistry is one relating to amplifying and detecting very small amounts of nucleic acids using the polymerase chain reaction (PCR).

The PCR procedure involves such a scheme that a reaction for doubling a target DNA molecule per thermal cycle using a pair (two) of primers (a forward primer and a reverse primer) is repeated, typically 20 to 40 times, to achieve significant amplification of the target DNA; PCR is an indispensable technique for today's experiments in genetic engineering. At early times of the development of the PCR procedure, this technique was used only for the purpose of amplification and from the viewpoint of DNA quantitation, it simply quantified the amplified DNA within a sample that had completed the PCR reaction.

A defect with the performance of the PCR reaction is that the primers hybridize with a sequence that is not of interest, causing non-specific amplification of that sequence. This problem still exists even today despite the advances in the primer design technology. This problem of non-specific amplification is unavoidable as long as the sample used as a template contains sequences that are similar to the target sequence. Since non-specific amplification is unavoidable, to solve this problem, it is necessary to check to see if the amplified sequence is the target sequence or a non-target sequence.

At a later time, a real-time PCR apparatus capable of performing the PCR reaction while measuring fluorescence was developed and amplification of the DNA in the sample can now be detected with a fluorescent dye for a continuous period concurrent with the performance of the PCR reaction. In early times following the development of the real-time PCR apparatus, the amount of fluorescence that depended on the amount of nucleic acids was measured using an intercalator such as ethidium bromide, so it was simply the amount of nucleic acids in the sample that could be measured. Quantitation in a more strict sense of the term is now possible at the level of the copy number of the nucleic acid (DNA or RNA) amplified and this was only after the development of a probe having a reporter and a quencher (e.g., TaqMan (trademark) probe). Development of this probe has enabled checking to see if the amplified sequence is the target sequence or a non-target sequence: since the amount of nucleic acids during amplification by PCR at the exponential amplification phase depends on the copy number (quantity) of the nucleic acid molecule that was initially present in the sample before the start of PCR, use of that probe has made it possible to express the quantify (degree) of gene expression in numerical terms at the level of copy number.

Quantitation based on the copy number of a nucleic acid molecule using the probe having a reporter and a quencher depends on the specificities of the sequences of the amplification primers and the sequence of the probe for the sequence of the target nucleic acid molecule. In other words, the quantitation using the probe having a reporter and a quencher is characterized in that the target sequence is amplified by the PCR procedure on the basis of the sequence specificity of the amplification primers for the target sequence and the amplified target sequence is detected using the probe having specificity, to thereby elevate the detection sensitivity.

Currently, a method that is often used to quantify the amount of gene expression in a sample is by using the above-described probe in the RT-PCR procedure using a reverse transcriptase. This RT-PCR procedure is such that a product in which about several hundred thousand copies of the target gene expression product can be quantified from a sample that usually comprises 1 ng of RNA. However, it has been reported that even if the above-described type of probe which is capable of sequence-specific detection is used, the presence of a non-target gene in a nucleic acid molecule in the sample which is other than the target gene but has a similar sequence thereto, or the presence of a repeat sequence of the similar sequence, or the presence of a non-target gene having a family sequence formed by alternative splicing causes amplification of fragments of the non-target gene containing the non-target sequence, which results in noise to quantitation of the target nucleic acid molecule. Thus, in order to ensure that specific discrimination between such similar sequences and quantitation of the target gene are performed in high precision, it has been required in the technical field of interest to develop a new means for amplifying only the target gene specifically and precisely.

In particular, in medical fields such as tailor-made medicine and in many fields founded on life science, it has recently been required that very small amounts of an expressed gene (biosynthesized RNA) on the order of about several hundred copies should be quantified extremely rigorously and with high precision (in high sensitivity and specificity) and there has been a demand for a new technique having an enhanced degree of quantitation.

### DISCLOSURE OF THE INVENTION

### Problems to be Solved by the Invention

The present invention has an objective of developing a new means for amplifying a target gene with high sensitivity on the basis of specific sequence discrimination between similar sequences. The present invention has another objective of developing a new means for precise quantitation of a target gene on the basis of specific sequence discrimination between similar sequences. Specifically, the present invention has objectives of developing a method of reducing the noise from a non-target gene having a similar sequence so that only a target gene is specifically amplified, and of developing a new method of quantitation that involves acquiring a signal from the amplified sequence with high precision (in high sensitivity and specificity).

### Means for Solving the Problems

As a result of the intensive studies they conducted, the present inventors found that the above-mentioned problems could be solved by incorporating one or more types of a third primer in a PCR reaction mixture (this third primer is hereinafter referred to as “an intercept primer”) and the present invention has accordingly been accomplished.

Specifically, the present invention is characterized by providing a method for sensitive amplification of a nucleic acid having a target sequence, which is characterized by performing a nucleic acid amplification reaction in a sample containing the target sequence and a non-target sequence similar thereto using:

a first primer and a second primer that amplify the target sequence but which are also capable of amplifying the non-target sequence; and

one or more intercept primers comprising a sequence that does not hybridize with the target sequence but hybridizes with the non-target sequence, wherein:

part of the non-target sequence is amplified with the one or more intercept primers and the first primer, whereby a non-target sequence portion which is deficient of the site on the non-target sequence that is to be recognized by the second primer is generated and non-specific amplification of the non-target sequence due to the pair of the first and second primers is reduced; and

an amplification product derived from the target sequence as amplified with the first and second primers is selectively obtained.

In another aspect, the present invention is characterized by providing a method for sensitive measurement of the expression of a nucleic acid having a target sequence, which is characterized by performing a quantitative nucleic acid amplification reaction in a sample containing the target sequence and a non-target sequence similar thereto using:

a first primer and a second primer that amplify the target sequence but which are also capable of amplifying the non-target sequence;

one or more intercept primers comprising a sequence that does not hybridize with the target sequence but hybridizes with the non-target sequence; and

a target sequence recognizing probe that recognizes the target sequence, wherein;

part of the non-target sequence is amplified with the one or more intercept primers and the first primer, whereby a non-target sequence portion which is deficient of the site on the non-target sequence that is to be recognized by the target sequence recognizing probe is generated and a detection signal derived from the non-target sequence by the target sequence recognizing probe is reduced; and

a signal derived from the target sequence as amplified with the first and second primers is selectively obtained using the target sequence recognizing probe.

In yet another aspect, the present invention is characterized by providing a method for measuring the ratio between the amounts of the expression products of the target sequence and the non-target sequence in a sample, which is characterized by performing a quantitative nucleic acid amplification reaction in the sample containing the target sequence and the non-target sequence similar thereto using;

a first primer and a second primer that amplify the target sequence but which are also capable of amplifying the non-target sequence;

one or more intercept primers comprising a sequence that does not hybridize with the target sequence but hybridizes with the non-target sequence;

a first recognition probe that recognizes the target sequence; and

a second recognition probe that recognizes the non-target sequence but does not recognize the target sequence, wherein:

part of the non-target sequence is amplified with the one or more intercept primer and the first primer to thereby generate a non-target sequence portion which is deficient of the site on the non-target sequence that is to hybridize with the first recognition probe; and

a first signal as obtained using the first recognition probe and derived from the target sequence as amplified with the first and second primers is compared with a second signal as obtained using the second recognition probe and derived from the non-target sequence portion, whereby the ratio between the amounts of the expression products of the target sequence and the non-target sequence in the sample is measured.

In a still further aspect, the present invention is characterized by providing a method for simultaneously measuring the amounts of the expression products of the target sequence and the non-target sequence in a sample, which is characterized by performing a quantitative nucleic acid amplification reaction in the sample containing the target sequence and the non-target sequence similar thereto using:

a first primer and a second primer that amplify the target sequence but which are also capable of amplifying the non-target sequence;

one or more intercept primers comprising a sequence that does not hybridize with the target sequence but hybridizes with the non-target sequence;

a first recognition probe that recognizes the target sequence; and

a second recognition probe that recognizes the non-target sequence but does not recognize the target sequence, wherein:

part of the non-target sequence is amplified with the one or more intercept primers and the first primer to thereby generate a non-target sequence portion which is deficient of the site on the non-target sequence that is to hybridize with the first recognition probe; and

a first signal as obtained using the first recognition probe and derived from the target sequence as amplified with the first and second primers is compared with a second signal as obtained using the second recognition probe and derived from the non-target sequence portion, whereby the ratio between the amounts of the expression products of the target sequence and the non-target sequence in the sample is measured.

### EFFECTS OF THE INVENTION

The present invention enables sensitive and specific amplification of the target sequence, measurement of the amount of expression product of the target sequence with high precision (in high sensitivity and specificity), measurement of the ratio between the amounts of the expression products of the target sequence and the non-target sequence, and simultaneous quantitation of the amounts of the expression products of the target sequence and the non-target sequence in the sample with high precision (in high sensitivity and specificity), using the three types of amplification primers (i.e., the first primer, the second primer, and the intercept primer).

Each aspect of the present invention can be realized by simply mixing at least one intercept primer as the third primer into the nucleic acid amplification reaction mixture. Hence, it has such a feature that the present invention can be applied to any of the conventional techniques for nucleic acid amplification reaction (e.g., the PCR procedure, the RT-PCR procedure, the LAMP procedure, or multiplex PCR). It also has such a feature that the present invention can be realized using any of the conventional means for real-time detection of nucleic acid amplification (e.g., the TaqMan probe procedure or the FERT procedure). Hence, without installing any new facilities or equipment but using the conventionally used facilities or equipment, the present invention makes it possible to amplify or quantitate the amplified target sequence with precision 10 to 100 times higher than methods that have been performed conventionally, or to measure the amounts of the amplified sequence(s) based on that precise quantitation.

### BRIEF DESCRIPTION OF THE DRAWINGS

FIG. 1 is a diagram showing the concept of the method of the present invention for sensitive amplification using the three types of primers, the first primer, the second primer, and the Intercept primer.

FIG. 2 is a diagram showing the concept of the method of the present invention for sensitive measurement using a target sequence recognizing probe in addition to the three types of primers, the first primer, the second primer, and the intercept primer.

FIG. 3 is a diagram showing the concept of a method for sensitive discrimination of human 3Rtau and 4Rtau using the three types of primers, the first primer, the second primer, and the intercept primer.

FIG. 4 is a diagram showing that noise from non-specific amplification of a non-target sequence is reduced by using the three types of primers, the first primer, the second primer, and the intercept primer.

FIG. 5 shows that the amplification of a target sequence by the pair of the first and second primers using a human 3Rtau encoding nucleic acid molecule as a template is not affected by the presence or absence of the intercept primer.

FIG. 6 shows that the presence of the intercept primer enables only a target sequence to be amplified specifically from a mixture of the target sequence and a non-target sequence and that the amplification of the target sequence is not affected by the amount of the non-target sequence added to the sample.

FIG. 7 shows that a target sequence and a non-target sequence can be quantified simultaneously by using a sample containing a mixture of the target and non-target sequences as a template and using a target sequence recognizing probe and a non-target sequence recognizing probe simultaneously in the presence of the intercept primer.

### MODES FOR CARRYING OUT THE INVENTION

In its first aspect, the present invention provides a method for sensitive amplification of a nucleic acid having a target sequence, which is characterized by performing a nucleic acid amplification reaction in a sample containing the target sequence and a non-target sequence similar thereto, using the three types of primers, a first primer and a second primer that amplify the target sequence but which are also capable of amplifying the non-target sequence, and an intercept primer comprising a sequence that does not hybridize with the target sequence but hybridizes with the non-target sequence.

This method is characterized in that part of the non-target sequence is amplified with the intercept primer and the first primer to thereby generate a non-target sequence portion which is deficient of the site on the non-target sequence that is to be recognized by the second primer. By using the intercept primer, non-specific amplification of the non-target sequence due to the pair of the first and second primers can be reduced. As a result of this, a specific amplification product derived from the target sequence as amplified with the first and second primers can be selectively obtained.

Known as a method to reduce non-specific amplification while allowing specific amplification to be performed selectively in the nucleic acid amplification reaction is the method disclosed in JP 2003-159100 A. The method described in that patent document was developed as a method to detect single nucleotide polymorphisms and it Involves preparing a block primer, a primer specific to the nucleotide variation in a single nucleotide polymorphism and ensuring, based on the hybridization properties of the block polymer, that a template sequence having the nucleotide variation in polymorphism will not undergo non-specific amplification. This method is characterized by such features that it simply reduces the amplification of the variant template having the single nucleotide polymorphism and that use of the LAMP procedure (Notomi et al., Nucleic Acids Research (2000), Vol. 28, e63 and WO 00/28082) is an essential requirement to meet.

In contrast, the method of the present invention which uses the intercept primer is characterized in that the types of nucleic acid amplification reactions to which the intercept primer can be applied are not limited but that the intercept primer is used to positively perform non-specific amplification of the non-specific target. By adopting this design, not only in the case of a nucleotide sequence having single nucleotide polymorphisms but also many other target sequences where non-specific amplification of non-target sequences occurs in the conventional amplification method, the target sequences can be specifically and selectively amplified.

The “target sequence” as used herein refers to the sequence of a target gene which is to be amplified with the first and the second primer, and the “non-target sequence” refers to the sequence of a non-target gene which is to be amplified with the same pair of primers.

In the conventional nucleic acid amplification methods (e.g., the PCR procedure), amplification reaction has been carried out with two primers added to the sample that hybridize with the template (which correspond to the first and second primers in the present invention). However, if the sample contains gene expression products having various sequences, the cases where the undesired non-target sequence is amplified non-specifically whereas the desired target sequence is not amplified have frequently been occurred in the conventional nucleic acid amplification methods, as applied to perform the nucleic acid amplification reaction using the first and the second primer. The method of the present invention shall be applied to cases where such non-specific amplification takes place.

The present invention is characterized in that in addition to the aforementioned two types of primers (the first and the second primer), one or more types of an intercept primer as a third primer are also added to the sample for performing the amplification reaction. Hence, the present invention starts with designing the intercept primer to be used.

A nucleic acid amplification reaction is performed on a sample using the first and the second primer as primers. If amplification of an undesired sequence (i.e., a non-target sequence) is verified, the amplified target and non-target sequences are determined by DNA sequencing and compared those sequences with each other. On the basis of this sequence analysis, the intercept primer is designed in such a way that it hybridizes with the non-target sequence but not with the target sequence and that the pair of the first primer and the intercept primer will amplify a non-target sequence portion which is deficient of the site on the non-target sequence that is to be recognized by the second primer. By combining the intercept primer having those features with the above-mentioned first primer, the target sequence cannot be amplified but part of the non-target sequence can be amplified.

Here, the intercept primer is not particularly limited by its backbone structure but one having any backbone structure may be used considering the enhancement of affinity for the target nucleic acid and other characteristics such as an increase in stability in the presence of a nuclease. In the present invention, any intercept primers may be adopted that have the ribonucleic acid (RNA) backbone, deoxyribonucleic acid (DNA) backbone, or other backbones such as the peptide nucleic acid (PNA) backbone having the sugar backbone of an oligonucleotide replaced by an amide-containing backbone.

The thus designed first primer, second primer and intercept primer are added to the single nucleic acid amplification reaction mixture and a nucleic acid amplification reaction is performed on the aforementioned sample mixture. In this nucleic acid amplification reaction, a non-target sequence portion is amplified by the pair of the first primer and the intercept primer; however, since the amplified non-target sequence portion has no sequence that hybridizes with the second primer, the subsequent nucleic acid amplification reaction which uses the non-target sequence portion as a template cannot involve any amplification reaction that is initiated by the pair of the first and second primers.

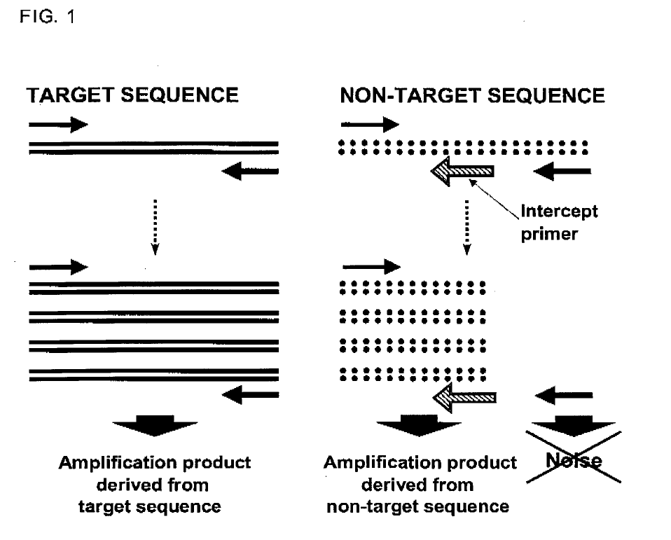

On the other hand, the target sequence is amplified by the pair of the first and second primers whereas the intercept primer does not hybridize with the target sequence, so the pair of the first primer and the intercept primer will not induce any non-specific amplification that uses the target sequence as a template. Consequently, non-specific amplification of the non-target sequence by the first and second primers can be cancelled. This aspect of the present invention which uses one intercept primer is conceptually depicted in FIG. 1.

If, in the case of performing a nucleic acid amplification reaction using the first and second primers as primers, amplification of more than one unwanted sequence (i.e., a plurality of non-target sequences) is verified, a plurality of intercept primers may be prepared and used within the same sample. Thus, in the method of the present invention, a plurality of intercept primers may be used. In the case of using a plurality of intercept primers, the aforementioned procedure for using one intercept primer may also be employed to design and prepare a plurality of intercept primers, and the first primer, the second primer and the prepared plurality of intercept primers are added to the single nucleic acid amplification reaction mixture, with a nucleic acid amplification reaction being performed on the aforementioned sample.

The sensitive amplification method of the present invention may be applied to any of the nucleic acid amplification reactions that are employed in the technical field concerned and such nucleic acid amplification reactions include, but are not limited to, the polymerase chain reaction (PCR), the RT-PCR procedure, the LAMP (loop-mediated isothermal amplification) procedure, and multiplex PCR.

In the case where the method of the present invention is applied to the PCR procedure or the RT-PCR procedure, linear oligonucleotide primers (the first primer, the second primer, and the intercept primer) are used; in the case where it is applied to the LAMP (loop-mediated isothermal amplification) procedure, two primers associated with the first primer region (which, for convenience sake, are herein collectively referred to as the first primer), two primers associated with the second primer region (which, for convenience sake, are herein collectively referred to as the second primer), and two primers associated with the intercept primer region (which, for convenience sake, are herein collectively referred to as the intercept primer).

The method of the present invention may also be applied to the multiplex PCR procedure. Multiplex PCR is a procedure currently used in practice for localizing the site or sites of variation on a large gene in which one or more of variations scattered on it are known to be associated with disease, such as the dystrophin gene, and this procedure is characterized by performing PCR reactions simultaneously using multiple sets of primers. To be more specific, the dystrophin gene is a large gene that is as large as 2.4 Mbp in size and has as many as 79 exons, so it is not practical to detect the site or sites of variation by amplifying its full length and, instead, only a known site or sites of variation are amplified to check for the presence of any variation that is associated with muscular dystrophy. Therefore, in the case where the method of the present invention is applied to the multiplex PCR procedure, multiple sets of linear oligonucleotide primers (the first primer, the second primer, and the intercept primer) which are used in one set in the case where it is applied to the PCR procedure or the RT-PCR procedure are used, whereby several runs of the nucleic acid amplification reaction can be performed simultaneously within the single reaction sample.

According to the second aspect of the present invention, the present invention may provide a method for sensitive measurement of the expression of a nucleic acid having a target sequence, where a recognition probe is further used as an additional component in the above-described method of sensitive amplification to perform a quantitative nucleic acid amplification reaction in a sample containing the target sequence and a non-target sequence. This aspect of the present invention can be applied to any of the quantitative nucleic acid amplification reactions that can amplify a nucleic acid molecule while quantitatively measuring the copy number of the nucleic acid molecule. Such quantitative nucleic acid amplification reactions include, but are not limited to, quantitative polymerase chain reaction (PCR), the RT-PCR procedure, the quantitative LAMP (loop-mediated isothermal amplification) procedure, and multiplex PCR.

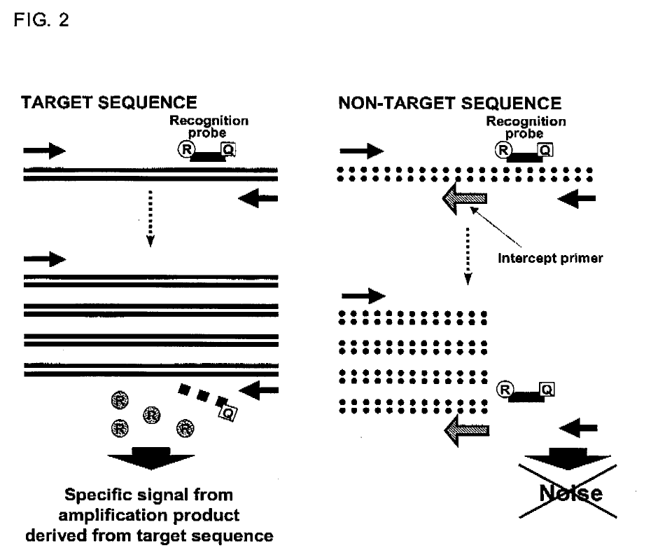

This aspect of the present invention is characterized in that not only the three types of primers, the first primer, the second primer, and at least one intercept primer, as in the aforementioned method for sensitive amplification, but also a recognition probe that recognizes the target sequence (i.e., a target sequence recognizing probe) are used. Hence, in this aspect of the present invention, not only the primers but also the recognition probe are designed. This aspect of the present invention which uses a single intercept primer is conceptually shown in FIG. 2.

The target sequence recognizing probe is designed in such a way that it recognizes a sequence that exists on the target sequence to be amplified by the first and the second primer but which does not exist on part of the non-target sequence to be amplified by the intercept primer and the first primer. The site to be recognized by the target sequence recognizing probe is preferably selected to occur, for example, in a target sequence portion that corresponds to the non-target sequence portion between the intercept primer and the second primer (namely, that portion which is not amplified by the intercept primer and the first primer) or at the site of splice junction used in alternative splicing.

When a quantitative nucleic acid amplification reaction is to be carried out, a nucleic acid amplification reaction in which the amplification of a nucleic acid molecule in the reaction mixture can be detected real-time, for example, a nucleic acid amplification method capable of real-time quantitation, is employed. Designed and prepared as target sequence recognizing probes for use in such nucleic acid amplification reaction capable of real-time quantitation are those probes that are commonly employed in that method and which use non-radioactive labeling compounds or the like to enable, for example, fluorescent detection, as exemplified by probes having both a reporter and a quencher, including TaqMan (trademark) probe (Applied Biosystems), QuantiTect (trademark) Probes (QIAGEN), QIAGEN Operon (Trademark) Dual-Labeled Probes (QIAGEN), FRET hybridization probes (e.g. LightCycler (trademark) Hybridization (FRET) Probe (Roche Diagnostics)), and Molecular Beacons (trademark) (Invitrogen). As a result of amplifying the target sequence and the non-target sequence using those three types of primers and detecting the amplified sequence using the target sequence recognizing probe, only the signal derived from the target sequence by virtue of the first and second primers can be detected while canceling the noise from non-specific amplification of the non-target sequence.

The present invention in its third aspect provides a method for measuring the ratio between the amounts of the expression products of the target sequence and the non-target sequence. When implementing this aspect of the present invention, two recognition probes, i.e., the above-mentioned target sequence recognizing probe (the first recognition probe) and a non-target sequence recognizing probe (the second recognition probe), are incorporated in the nucleic acid amplification reaction mixture. In this case, the non-target sequence recognizing probe is designed and prepared in such a way that it hybridizes with the sequence on part of the non-target sequence which is to be amplified by the pair of the first primer and the intercept primer but does not hybridize with the sequence on the target sequence which is to be amplified by the pair of the first and the second primer.

Therefore, in the third aspect of the present invention, the above-described three types of amplification primers (i.e., the first primer, the second primer, and one or more intercept primers) as well as two types of recognition probes (the target sequence recognizing probe (the first recognition probe) and the non-target sequence recognizing probe (the second recognition probe)) are used to carry out a nucleic acid amplification reaction such as RT-PCR or PCR reaction while measuring fluorescence real-time with a nucleic acid amplification reaction apparatus capable of real-time quantitation.

Suppose here the case where the target gene has an initial copy number of A, the curve for its amplification rises exponentially at n cycles, a non-target gene has an initial copy number of B, and the curve for its amplification rises exponentially at k cycles. The signal from the first recognition probe at n cycles that serves as an index for amplification of the target sequence theoretically means correspondence to A×2ⁿ⁻¹ copies of the target sequence, whereas the signal from the second recognition probe at k cycles that serves as an index for amplification of the non-target sequence theoretically means correspondence to B×2^{k-1} copies of the target sequence. At the point in time where the curve for amplification of a certain nucleic acid molecule rises exponentially, the copy number of the nucleic acid molecule that is contained can be presumed to be constant, so the equation of A×2ⁿ⁻¹=B×2^{k-1} holds theoretically, hence A/B=2^{k-n} holds theoretically.

Therefore, by comparing the signal obtained from the first recognition probe with the signal obtained from the second recognition probe and identifying the difference in the number of cycles between the points where the two curves for amplification rise exponentially (i.e., the value of k−n), one can measure the ratio between the target and non-target genes contained in the initial sample as A/B=2^{k-n}.

The present invention in its fourth aspect provides a method for simultaneously measuring the amounts of the expression products of the target sequence and the non-target sequence within a single sample. In this aspect, the above-described three types of amplification primers (i.e., the first primer, the second primer, and one or more intercept primers) as well as two types of recognition probes (the target sequence recognizing probe (the first recognition probe) and the non-target sequence recognizing probe (the second recognition probe)) are used to carry out a nucleic acid amplification reaction such as RT-PCR or PCR reaction while measuring fluorescence real-time with a nucleic acid amplification reaction apparatus capable of real-time quantitation. Addition of the intercept primer inhibits non-specific amplification of a DNA fragment derived from a non-target nucleic acid molecule by the pair of the first and second primers and, hence, noise derived from hybridization of the target sequence recognizing probe with the non-target sequence can be reduced, with the result that the amount of expression product of the target gene can be measured with high precision (in high sensitivity and specificity). On the other hand, the second recognition probe is capable of hybridizing with the non-target sequence and, hence, specific amplification of the non-target nucleic acid molecule by the pair of the first primer and the intercept primer can be detected. As a result of this, the amount of expression product of the non-target gene can be measured with high precision (in high sensitivity and specificity). In this way, the present invention also enables the amounts of the expression products of the target sequence and the non-target sequence within a sample to be measured simultaneously with high precision (in high sensitivity and specificity).

In any of the aspects of the present invention, one may perform a nucleic acid amplification reaction using cDNA or genomic DNA as a template or one may perform the RT-PCR procedure in which nucleic acid amplification is preceded by a reverse transcriptase reaction using RNA.

On the pages that follow herein, examples are described with a view to explaining the invention in greater detail. It should, however, be understood that the descriptions of those examples are by no means intended to limit the scope of the present invention but are merely intended to explain the invention in greater detail.

### EXAMPLES

### Example 1

### Designing Amplification Primers and Recognition Probes for a Nucleic Acid Molecule Coding for Human 3 Repeat Tau and a Nucleic Acid Molecule Coding for Human 4 Repeat Tau

In this example, a sample containing both a target gene and a non-target gene was subjected to a nucleic acid amplification reaction, with an intercept primer being used so as to amplify and quantitate the target sequence specifically.

Stated specifically, this example used a sample containing both a nucleic acid molecule coding for the target sequence, human 3 repeat tau (3Rtau) (NCBI: NM_{—}016841 (5464 bp)), and a nucleic acid molecule coding for the non-target sequence, human 4 repeat tau (4Rtau) (NCBI: NM_{—}016834 (5557 bp)), and it had an objective of reducing the noise derived from the 4Rtau encoding nucleic acid molecule so as to amplify and quantify the 3Rtau encoding nucleic acid molecule in a specific manner. To attain this object, amplification primers and recognition probes were designed for the human 3Rtau encoding nucleic acid molecule and the human 4Rtau encoding nucleic acid molecule.

Human 3Rtau and 4Rtau are two proteins that are produced from a single gene by alternative splicing and which are associated with the pathology of Alzheimer's disease. In humans, dementia-involving diseases are known that are characterized by abnormal accumulation of tau gene products in degenerative nerve cells, and examples of the diseases include Alzheimer's disease, frontotemporal dementia that involves Parkinsonian syndrome linked to chromosome 17 (FTD P-17), Pick's disease, progressive supranuclear palsy (PSP), and corticobasal degeneration (CBD). Each of 3Rtau and 4Rtau is recognized as a major constituent of a structure that has accumulated abnormally within degenerative nerve cells and whichever of 3Rtau and 4Rtau will accumulate varies from one disease to another. For instance, it is known that both 3Rtau and 4Rtau accumulate in Alzheimer's disease but that only 3Rtau accumulates in Pick's disease whereas only 4Rtau accumulates in familial mutation of FTD P-17, PSP, and CBD. Therefore, precise differential quantitation of the amounts of expression products of two nucleic acid molecules, one coding for 3Rtau and the other coding for 4Rtau, is very important from the viewpoint of studying the pathologies of diseases in which the tau gene is involved.

These nucleic acid molecules, one coding for 3Rtau and the other coding for 4Rtau, are generated by alternative splicing of RNA transcribed from the same genomic region. Stated specifically, the nucleic acid molecule coding for human 3Rtau undergoes splicing between exons 9 and 11 in the tau gene region and eventually has a structure that consists of sequence without an exon 10 (i.e., ˜exon 9˜exon 11˜; SEQ ID NO:1). On the other hand, the nucleic acid molecule coding for 4Rtau undergoes splicing in such a way that exon 10 is present between exons 9 and 11 and it eventually has a structure that consists of sequence containing an exon 10 (i.e., ˜exon 9˜exon 10˜exon 11˜; SEQ ID NO:2). Hence, if the first primer is set on exon 9 and the second primer on exon 11, two sequences are amplified to yield an amplification product derived from the 3Rtau encoding nucleic acid molecule that contains a sequence derived from exon 11, as well as an amplification product derived from the 4Rtau encoding nucleic acid molecule that contains the sequence of exon 10 and a sequence derived from exon 11; this makes it difficult to achieve specific amplification of the nucleic acid molecule that codes for 3Rtau.

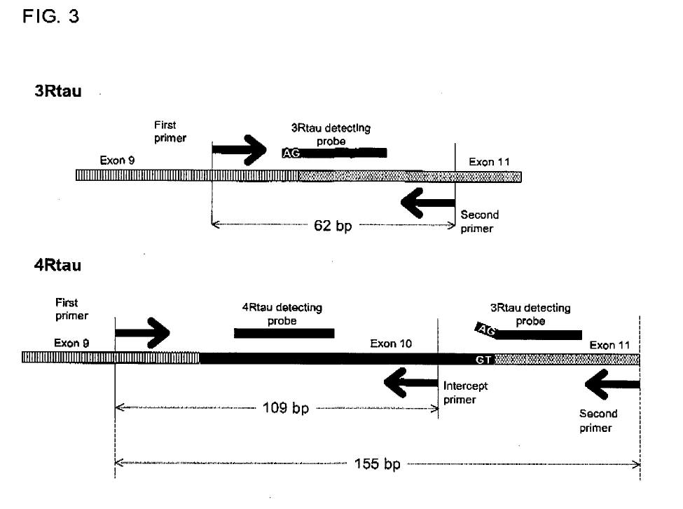

In Example 1, two primers for amplifying the human tau gene were designed and prepared; they were the first primer derived from the sense strand sequence of exon 9 in the human tau gene and designated htauR1_F:5′-gaacctgaagcaccagccg-3′ (SEQ ID NO:3), and the second primer derived from the antisense strand of exon 11 in the human tau gene and designated htauR3_R:5′-tcaggtcaactggtttgt-3′ (SEQ ID NO:4) (Hokkaido System Science Co., Ltd.). Using these primers, one can amplify both a human 3Rtau encoding nucleic acid molecule (62 bp) containing a sequence derived from exon 11 and a human 4Rtau encoding nucleic acid molecule (155 bp) containing the sequence of exon 10 and a sequence derived from exon 11 (FIG. 3).

At the same time, an intercept primer was constructed: being designated htauR2_R:5′-gacgtgtttgatattatcctttg-3′ (SEQ ID NO:5), this primer was derived from the antisense strand of exon 10 and would not hybridize with the human 3Rtau encoding nucleic acid molecule but hybridize with the human 4Rtau encoding nucleic acid molecule. By using this primer in combination with the aforementioned first primer (htauR1_F) derived from the sense strand of exon 9 in the human tau gene, the sequence derived from exon 9˜exon 10 (109 bp) in the human 4Rtau encoding nucleic acid molecule can be amplified but the nucleic acid molecule containing the sequence derived from exon 11 in human tau cannot (FIG. 3).

In Example 1, a probe having a reporter and a quencher was also designed and prepared; being designated 3RVIC:5′-aggtgcaaatagtct-3′ (SEQ ID NO:6), this probe (TaqMan (trademark) MGB probe, Applied Biosystems) had the reporter VIC (trademark) and a quencher and would recognize the human 3Rtau encoding nucleic acid molecule (62 bp) containing the sequence derived from exon 11. This probe 3RVIC was so constructed that it would hybridize with the junction region between exons 9 and 11 in the human 3Rtau encoding nucleic acid molecule, and it can specifically recognize the human 3Rtau encoding nucleic acid molecule (62 bp) containing the sequence derived from exon 11 (FIG. 3).

In Example 1, another probe having a reporter and a quencher was further designed and prepared; being designated 4RFAM:5′-tagcaacgtccagtcca-3′ (SEQ ID NO:7), this probe (TaqMan (trademark) MGB probe, Applied Biosystems) had the reporter FAM (trademark) and a quencher and would recognize the human 4Rtau encoding nucleic acid molecule. This probe 4RFAM was so constructed that it would hybridize with exon 10 in the human 4Rtau encoding nucleic acid molecule, and it was so designed that it could recognize part of the human 4Rtau encoding nucleic acid molecule that would be amplified by the pair of the aforementioned first primer (htauR1_F) and intercept primer (htauR2_R). On the other hand, this probe 4RFAM will not recognize the human 3Rtau encoding nucleic acid molecule since the recognition site of this probe has no corresponding sequence on the human 3Rtau encoding nucleic acid molecule that will be amplified using the pair of the first primer (htauR1_F) and the second primer (htauR3_R) (FIG. 3).

### Example 2

### Reducing the Noise Derived from 4Rtau Encoding Nucleic Acid Molecule Using the Intercept Primer

In this example, a nucleic acid amplification reaction was performed using the intercept primer, with the human 4Rtau encoding nucleic acid molecule being used as a template, in order to verify an effect of the intercept primer to reduce the noise derived from the human 4Rtau encoding nucleic acid molecule.

In the test group, amplification was performed using the three types of primers already described in Example 1, i.e., the first primer, the second primer, and the intercept primer, as amplification primers; in the control group, amplification was performed using the two primers, i.e. the first and second primers, as amplification primers. In Example 2, the probe 3RVIC that would recognize the amplified human 3Rtau encoding nucleic acid molecule (62 bp) containing the sequence derived from exon 11 was used as a recognition probe to perform real-time PCR.

The PCR reagents were obtained from QIAGEN. A tau gene sequence containing vector was transfected to SHSY-5Y cells derived from neuroblastoma, which originally express sufficiently low level of the tau, to generate forced expression of the tau gene in the transfected cells. A PCR reaction was executed using a tau mRNA containing total RNA (1 μg) obtained from the transfected cells as a template for 4Rtau and a final volume of 25 μL of a PCR cocktail (0.2 μM htauR1_F primer, 0.20M htauR3_R primer, and 0.4 μM htauR2_R primer, as well as 0.1 μM 3RVIC probe, and 2× QuantiTect (trademark) Probe RT-PCR Master Mix (QIAGEN)). The PCR reaction consisted of 50° C.×30 min incubation for reverse transcription reaction, followed by 95° C.×15 min incubation for inactivation of the reverse transcriptase and activation of HotStar (trademark) Taq DNA polymerase (QIAGEN), which in turn was followed by 40 cycles of a 2-step PCR protocol (95° C.×15 sec (for denaturation), followed by 60° C.×1 min (for annealing/extension), all these steps being performed using ABI PRISM 7700 or 7300 (both being products of Applied Biosystems).

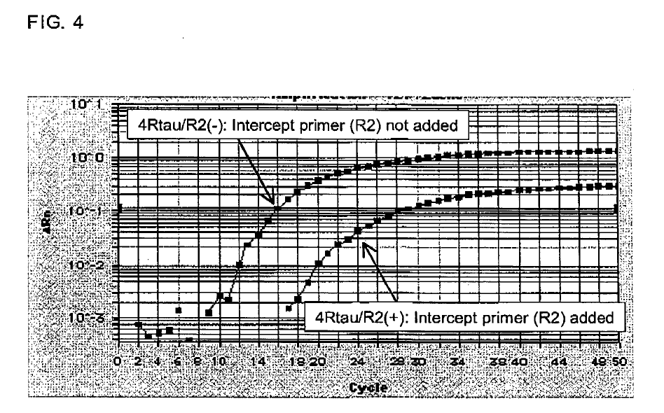

The results are shown in FIG. 4. As this Figure shows, in the control group containing no intercept primer, the fluorescence derived from the 3RVIC probe was such that the amplification curve rose exponentially at 10-11 cycles (4Rtau/R2(−) in FIG. 4), whereas in the test group containing the intercept primer in the PCR reaction mixture, it became clear that the sequence that was detected with the 3RVIC probe capable of recognizing the human 3Rtau encoding nucleic acid molecule (62 bp) containing the sequence derived from exon 11 could be reduced to a level almost comparable to that for the negative control (4Rtau/R2(+) in FIG. 4).

From these results, it became clear that in the control group where the first and second primers were used as amplification primers, with the human 4Rtau encoding nucleic acid molecule being used as a template, the sequence (155 bp) derived from the 4Rtau encoding nucleic acid molecule containing the sequence of exon 10 and the sequence derived from exon 11 was amplified as noise, but that by using the three types of primers, i.e., the first primer, the second primer and the intercept primer, on the same template, the noise sequence (155 bp) derived from the 4Rtau encoding nucleic acid molecule was no longer amplified, producing reduced noise.

### Example 3

### Effect of the Intercept Primer on Amplification of the Human 3Rtau Encoding Nucleic Acid Molecule

In this example, PCR was performed using the intercept primer in order to check to see if the intercept primer would cause any effect on amplification of the human 3Rtau encoding nucleic acid molecule.

A tau gene sequence containing vector was transfected to SHSY-5Y cells derived from neuroblastoma, which originally express sufficiently low level of the tau, to generate forced expression of the tau gene in the transfected cells. For experimentation, a nucleic acid amplification reaction was performed as described in Example 2 using a tau mRNA containing total RNA (1 μg) obtained from the transfected cells as a template for 3Rtau; in the test group, the above-mentioned three types of primers, i.e., the first primer, the second primer, and the intercept primer, were used as amplification primers, and in the control group, the two primers, i.e. the first and second primers.

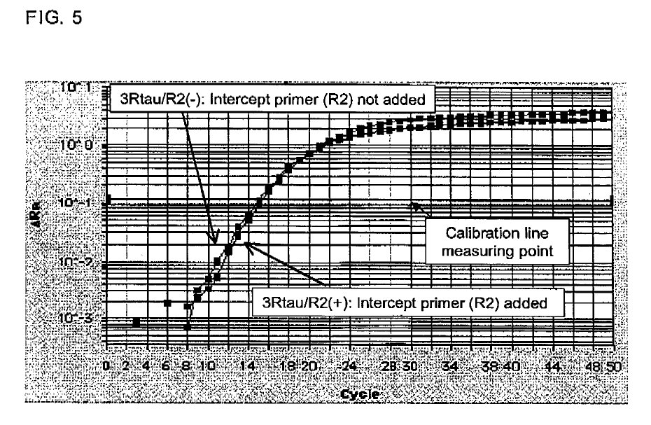

The results are shown in FIG. 5. As this Figure shows, it became clear that in both the test and control groups, the amplification curves rose exponentially at almost the same cycle number (3Rtau/R2(−) and 3Rtau/R2(+) in FIG. 5). From these results, it became clear that when the nucleic acid amplification reaction was performed using the human 3Rtau encoding nucleic acid molecule as a template, the amplification by the pair of the above-described first and second primers was in no way affected by the presence of the intercept primer in the reaction mixture.

### Example 4

### Specific Amplification of Human 3Rtau Encoding Nucleic Acid Molecule in the Presence of Intercept Primer

In this example, PCR was performed using the intercept primer in order to verify that the presence of the intercept primer caused specific amplification of the human 3Rtau encoding nucleic acid molecule to occur.

For experimentation, two samples of total RNA were prepared, one of which was total RNA containing the human 3Rtau encoding nucleic acid molecule derived from COST cells which was obtained by forcibly expressing 3Rtau, and the other of which was total RNA containing the human 4Rtau encoding nucleic acid molecule derived from COS7 cells which was obtained by forcibly expressing 4Rtau. These two samples of total RNA were mixed in three groups, based on the amount of total RNA, at the following mixing ratios of 3Rtau/4Rtau: 10 ng/0 ng in group A; 10 ng/10 ng in group B; and 10 ng/50 ng in group C; each of these mixtures was used as a template. The three types of primers already described in Example 1, i.e., the first primer (0.4 μM), the second primer (0.4 μM), and the intercept primer (0.8 μM), were used as amplification primers, and a nucleic acid amplification reaction was performed as described in Example 2. In that nucleic acid amplification reaction, 0.1 μM of the 3RVIC probe was used to detect the amplification products.

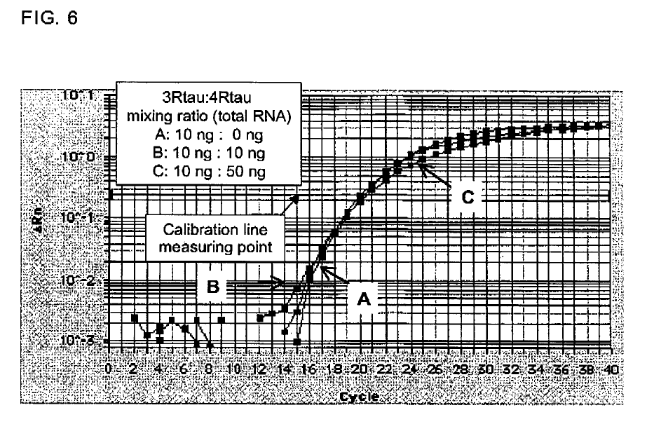

The results are shown in FIG. 6. As this figure shows, it became clear that in whichever of the mixtures of groups A to C was used as the template, the 3Rtau encoding nucleic acid (62 bp) containing the sequence derived from exon 11 was amplified at virtually the same rate (FIG. 6). Therefore, on account of the presence of the intercept primer, only the target sequence human 3Rtau encoding nucleic acid molecule (62 bp) containing the sequence derived from exon 11 could be specifically amplified from the mixture of the human 3Rtau encoding nucleic acid molecule and the human 4Rtau encoding nucleic acid molecule and the amplification of that human 3Rtau encoding nucleic acid molecule containing the sequence derived from exon 11 was in no way affected by the amount of the 4Rtau encoding nucleic acid molecule that had been added to the sample.

### Example 5

### Simultaneous Quantitation of a Mouse 3Rtau Encoding Nucleic Acid Molecule and a Mouse 4Rtau Encoding Nucleic Acid Molecule Using the Intercept Primer

In this example, PCR was performed using the intercept primer in order to verify that a mouse 3Rtau encoding nucleic acid molecule and a mouse 4Rtau encoding nucleic aid molecule could be quantified simultaneously.

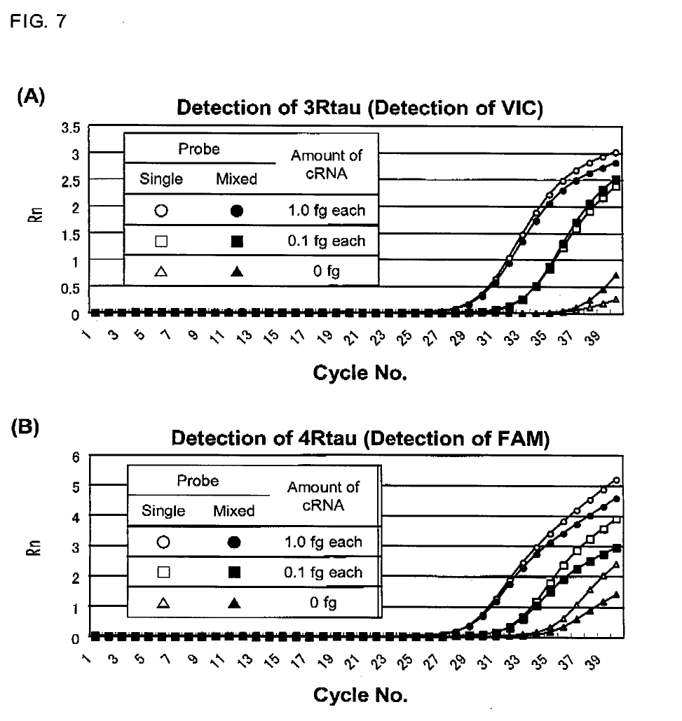

Mouse cDNA (3Rtau and 4Rtau) were subcloned into plasmids, which were used as templates for cRNA synthesis with an in vitro transcription reaction kit (RiboMAX™ Large scale RNA production system-T7: Promega). The cRNA of 3Rtau and the cRNA of 4Rtau (each weighing 1.0 fg in FIG. 7A and 0.1 fg in FIG. 7B) were mixed at a mass ratio of 1:1 to prepare a template for subsequent quantitation by RT-PCR.

In Example 5, the three types of primers, the first primer, the second primer and the intercept primer, were designed and prepared as primers for amplifying the mouse tau gene (Hokkaido System Science Co., Ltd.); the first primer was derived from the sense strand sequence of exon 9 in the mouse tau gene and designated MMtauR1_F:5′-gaacctgaagcaccagcca-3′ (SEQ ID NO:8); the second primer was derived from the antisense strand of exon 11 in the mouse tau gene and designated MMtauR3_R:5′-tcaggtccaccggcttgt-3′ (SEQ ID NO:9); and the intercept primer was derived from the antisense strand of exon 10 that did not exist in the nucleic acid molecule coding for mouse 3Rtau but which existed in the nucleic acid molecule coding for mouse 4Rtau and it was designated MMtauR2_R:5′-gacgtgtttgatattatccttcg-3′ (SEQ ID NO:10).

The experiment was conducted on six divided groups, which are indicated by ◯, , □, ▪, Δ and ▴, respectively (see FIG. 7A and FIG. 78).

In the experimental groups ◯ and , a 1:1 mixture of the cRNAs of 3Rtau and 4Rtau each weighing 1.0 fg was used as a template; in the experimental groups □ and ▪, a 1:1 mixture of the cRNAs of 3Rtau and 4Rtau each weighing 0.1 fg was used as a template; but in the experimental groups Δ and ▴, no template was contained in the reaction mixtures.

The following recognition probes were used: for ◯, □, and Δ in FIG. 7A, only 3RVIC probe (SEQ ID NO:6) (0.1 μM) was used; for ◯, □, and Δ in FIG. 7B, only 4RFAM probe (SEQ ID NO:7) (0.1 μM) was used; for , ▪, and ▴ in each of FIGS. 7A and 7B, both 3RVIC probe (SEQ ID NO:6) (0.1 μM) and 4RFAM probe (SEQ ID NO:7) (0.1 μM) were used. In FIG. 7A, only the fluorescence derived from 3RVIC probe was detected; in FIG. 7B, only the fluorescence derived from 4RFAM probe was detected.

To all groups, the above-mentioned three types of primers, i.e., the first primer (MMtauR1_F, 0.3 μM), the second primer (MMtauR3_R, 0.3 μM), and the intercept primer (MMtauR2_R, 0.6 NM), were added as amplification primers.

The other experimental conditions were the same as described in Example 2, and a nucleic acid amplification reaction was carried out.

The nucleic acid molecules used as a template in Example 5, one of which is 1.0 fg of the nucleic acid molecule coding for mouse 3Rtau and the other of which is 1.0 fg of the nucleic acid molecule coding for mouse 4Rtau, correspond to about 12000 to 15000 copies, whereas the nucleic acid molecules used as a template in the same Example, one of which is 0.1 fg of the nucleic acid molecules coding for mouse 3Rtau and the other of which is 0.1 fg of the nucleic acid molecules coding for mouse 4Rtau, correspond to about 1200 to 1500 copies. Therefore, a theoretical prediction is that the curves for amplification will differ by 3 to 4 cycles in the number of cycles at which they rise exponentially.

In FIG. 7A, ◯ and represent curves showing the increase of fluorescence derived from 3RVIC probe as obtained from the amplification products derived from 1.0 fg of 3Rtau cRNA, whereas □ and ▪ represent curves showing the increase of fluorescence derived from 3RVIC probe as obtained from the amplification products derived from 0.1 fg of 3Rtau cRNA.

As is clear from FIG. 7A, when 1.0 fg of 3Rtau cRNA was contained as a template, the signal originating from 3RVIC probe during amplification of the mouse 3Rtau encoding nucleic acid molecule (62 bp) containing the sequence derived from exon 11 rose exponentially at 31 cycles (◯ and in FIG. 7A), whereas when 0.1 fg of 3Rtau cRNA was contained as a template, the signal originating from 3RVIC probe during amplification of the mouse 3Rtau encoding nucleic acid molecule (62 bp) containing the sequence derived from exon 11 rose exponentially at 34 cycles (□ and ▪ in FIG. 7A). This result was independent of whether the samples contained only 3RVIC probe as a detection probe or they contained 3RVIC probe in admixture with 4RFAM probe.

In FIG. 75, on the other hand, ◯ and represent curves, showing the increase of fluorescence derived from 4RFAM probe as obtained from the amplification products derived from 1.0 fg of 4Rtau cRNA, whereas □ and ▪ represent curves showing the increase of fluorescence derived from 4RFAM probe as obtained from the amplification products derived from 0.1 fg of 4Rtau cRNA.

As is clear from FIG. 7B, when 1.0 fg of 3Rtau cRNA was contained as a template, the signal originating from 4RFAM probe during amplification of the mouse 4Rtau encoding nucleic acid molecule (109 bp) containing the sequence of exon 10 but not containing the sequence derived from exon 11 rose exponentially at 31 cycles (◯ and in FIG. 75) whereas when 0.1 fg of 4Rtau cRNA was contained as a template, the signal originating from 4RFAM probe during amplification of the mouse 4Rtau encoding nucleic acid molecule (109 bp) containing the sequence of exon 10 but not containing the sequence derived from exon 11 rose exponentially at 34 cycles (□ and ▪ in FIG. 7B). This result was independent of whether the samples contained only 4RFAM probe as a detection probe or they contained 4RFAM probe in admixture with 3RVIC probe.

This result shows that in proportion to the copy number of the human 3Rtau encoding nucleic acid molecule that was initially contained in the mixture at the start of the nucleic acid amplification reaction, the number of cycles at which the curves for the increase of fluorescence rose exponentially shifted (FIG. 7A) and the shift in cycle number was in agreement with the theoretically expected value (a shift of 3 or 4 cycles). A similar result was obtained in the case of the nucleic acid molecule coding for human 4Rtau (FIG. 7B).

The above-described observations show that by mixing the intercept primer in the PCR reaction mixture, the two nucleic acid molecules in the sample, one coding for human 3Rtau and the other coding for human 4Rtau, can be quantified simultaneously, as a result of which the ratio between the amounts of expression products of the two genes present in the sample can be measured.

### INDUSTRIAL APPLICABILITY

The present invention enables amplification of a nucleic acid having a target sequence with high precision (in high sensitivity and specificity), measurement of the amount of expression product of a target sequence with high precision (in high sensitivity and specificity), measurement of the ratio between the amounts of the expression products of the target sequence and the non-target sequence, and simultaneous quantitation of the amounts of the expression products of the target sequence and the non-target sequence in the sample with high precision (in high sensitivity and specificity) using the three amplification primers (i.e., the first primer, the second primer, and the intercept primer).

Each aspect of the present invention can be realized by simply mixing the intercept primer as the third primer into the nucleic acid amplification reaction mixture. Hence, it has such a feature that the present invention can be applied to any of the conventional techniques for nucleic acid amplification reaction (e.g., the PCR procedure, the RT-PCR procedure, the LAMP procedure, or multiplex PCR). It also has such a feature that the present invention can be realized using any of the conventional means for real-time detection of nucleic acid amplification (e.g., the TaqMan probe procedure or the FERT procedure). Hence, without installing any new facilities or equipment, the present invention makes it possible to quantitate the amplified target sequence with precision 10 to 100 times higher than the methods that has been performed conventionally, or to measure the amounts of the amplified target sequence(s) based on that precise quantitation.

1. A method for sensitive amplification of a nucleic acid having a target sequence, which is characterized by performing a nucleic acid amplification reaction in a sample containing the target sequence and a non-target sequence similar thereto using:
a first primer and a second primer that amplify the target sequence but which are also capable of amplifying the non-target sequence; and
one or more primers (intercept primers) comprising a sequence that does not hybridize with the target sequence but hybridizes with the non-target sequence, wherein:
part of the non-target sequence is amplified with the intercept primer and the first primer, whereby a non-target sequence portion which is deficient of the site on the non-target sequence that is to be recognized by the second primer is generated and non-specific amplification of the non-target sequence due to the pair of the first and second primers is reduced; and
an amplification product derived from the target sequence as amplified with the first and second primers is selectively obtained.

2. The method for sensitive amplification of a nucleic acid having a target sequence according to claim 1, which uses one or more intercept primers.

3. The method for sensitive amplification of a nucleic acid having a target sequence according to claim 1 or 2, wherein the nucleic acid amplification reaction is polymerase chain reaction (PCR), LAMP (loop-mediated isothermal amplification), reverse transcriptase polymerase chain reaction (RT-PCR), or multiplex PCR.

4. The method for sensitive amplification of a nucleic acid having a target sequence according to any one of claims 1 to 3, wherein the target sequence is 3Rtau and the non-target sequence is 4Rtau.

5. A method for sensitive measurement of the expression of a nucleic acid having a target sequence, which is characterized by performing a quantitative nucleic acid amplification reaction in a sample containing the target sequence and a non-target sequence similar thereto using:
a first primer and a second primer that amplify the target sequence but which are also capable of amplifying the non-target sequence;
a primer (intercept primer) comprising a sequence that does not hybridize with the target sequence but hybridizes with the non-target sequence; and
a target sequence recognizing probe that recognizes the target sequence, wherein:
part of the non-target sequence is amplified with the intercept primer and the first primer, whereby a non-target sequence portion which is deficient of the site on the non-target sequence that is to be recognized by the target sequence recognizing probe is generated and a detection signal derived from the non-target sequence by the target sequence recognizing probe is reduced; and
a signal derived from the target sequence as amplified with the first and second primers is selectively obtained using the target sequence recognizing probe.

6. The method for sensitive measurement of the expression of a nucleic acid having a target sequence according to claim 5, which uses one or more intercept primers.

7. The method for sensitive measurement of the expression of a nucleic acid having a target sequence according to claim 5 or 6, wherein the quantitative nucleic acid amplification reaction is polymerase chain reaction (PCR), LAMP (loop-mediated isothermal amplification), reverse transcriptase polymerase chain reaction (RT-PCR), or multiplex PCR.

8. The method for sensitive measurement of the expression of a nucleic acid having a target sequence according to any one of claims 5 to 7, wherein the target sequence is 3Rtau and the non-target sequence is 4Rtau.

9. A method for measuring the ratio between the amounts of the expression products of the target sequence and the non-target sequence in a sample, which is characterized by performing a quantitative nucleic acid amplification reaction in the sample containing the target sequence and the non-target sequence similar thereto using:
a first primer and a second primer that amplify the target sequence but which are also capable of amplifying the non-target sequence;
a primer (intercept primer) comprising a sequence that does not hybridize with the target sequence but hybridizes with the non-target sequence;
a first recognition probe that recognizes the target sequence; and
a second recognition probe that recognizes the non-target sequence but does not recognize the target sequence, wherein:
part of the non-target sequence is amplified with the intercept primer and the first primer to thereby generate a non-target sequence portion which is deficient of the site on the non-target sequence that is to hybridize with the first recognition probe; and
a first signal as obtained using the first recognition probe and derived from the target sequence as amplified with the first and second primers is compared with a second signal as obtained using the second recognition probe and derived from the non-target sequence portion, whereby the ratio between the amounts of the expression products of the target sequence and the non-target sequence in the sample is measured.

10. The method for measuring the ratio between the amounts of the expression products of the target sequence and the non-target sequence according to claim 9, which uses one or more intercept primers.

11. The method for measuring the ratio between the amounts of the expression products of the target sequence and the non-target sequence according to claim 9 or 10, wherein the quantitative nucleic acid amplification reaction is polymerase chain reaction (PCR), LAMP (loop-mediated isothermal amplification), reverse transcriptase polymerase chain reaction (RT-PCR), or multiplex PCR.

12. The method for measuring the ratio between the amounts of the expression products of the target sequence and the non-target sequence according to any one of claims 9 to 11, wherein the target sequence is 3Rtau and the non-target sequence is 4Rtau.

13. A method for simultaneously measuring the amounts of the expression products of the target sequence and the non-target sequence in a sample, which is characterized by performing a quantitative nucleic acid amplification reaction in the sample containing the target sequence and the non-target sequence similar thereto using:
a first primer and a second primer that amplify the target sequence but which are also capable of amplifying the non-target sequence;
a primer (intercept primer) comprising a sequence that does not hybridize with the target sequence but hybridizes with the non-target sequence;
a first recognition probe that recognizes the target sequence; and
a second recognition probe that recognizes the non-target sequence but does not recognize the target sequence, wherein:
part of the non-target sequence is amplified with the intercept primer and the first primer to thereby generate a non-target sequence portion which is deficient of the site on the non-target sequence that is to hybridize with the first recognition probe; and
a first signal as obtained using the first recognition probe and derived from the target sequence as amplified with the first and second primers is compared with a second signal as obtained using the second recognition probe and derived from the non-target sequence portion, whereby the ratio between the amounts of the expression products of the target sequence and the non-target sequence in the sample is measured.

14. The method for simultaneously measuring the amounts of the expression products of the target sequence and the non-target sequence according to claim 13, which uses one or more intercept primers.

15. The method for simultaneously measuring the amounts of the expression products of the target sequence and the non-target sequence according to claim 13 or 14, wherein the quantitative nucleic acid amplification reaction is polymerase chain reaction (PCR), LAMP (loop-mediated isothermal amplification), reverse transcriptase polymerase chain reaction (RT-PCR), or multiplex PCR.

16. The method for simultaneously measuring the amounts of the expression products of the target sequence and the non-target sequence according to any one of claims 13 to 15, wherein the target sequence is 3Rtau and the non-target sequence is 4Rtau.
